(19) United States
(12) Patent Application Publication
(10) Pub. No.: US 2011/0046100 A1
(43) Pub. Date: Feb. 24, 2011

(54) Texaphyrin solutions and pharmaceutical formulations

(75) Inventors: Norbert Purro, Los Gatos, CA (US); Hugo Madden, Palo Alto, CA (US); Suzanne Kretchmar Nguyen, Los Gatos, CA (US)

Correspondence Address: WILSON, SONSINI, GOODRICH & ROSATI, 650 PAGE MILL ROAD, PALO ALTO, CA, 94304-1050, US

(73) Assignee: PHARMACYCLICS, INC., Sunnyvale, CA (US)

(21) Appl. No.: 12/939,780

(22) Filed: Nov. 4, 2010

Related U.S. Application Data
(63) Continuation of application No. 11/241,549, filed on Sep. 30, 2005.

(51) Int. Cl. A61K 31/555; A61P 35/00

(52) U.S. Cl. 514/185

(57) ABSTRACT

A packaging system is described for a drug that provides protection from contamination, crystallization and/or degradation of the drug during storage of the system prior to its use. The packaging of the drug does not significantly absorb, react with, or otherwise adversely affect the therapeutic effectiveness of the drug or other excipients or components during storage of the system prior to its use. The packaging system is also used for preserving a drug, particularly a drug containing high-purity texaphyrin metal complexes, by providing a product packaging system that prevents, limits or otherwise controls degradation reactions that can result from exposure to oxygen and/or light.

### FIELD OF THE INVENTION

The methods, compositions and materials described herein are directed to the storage of pharmaceutical formulations.

### BACKGROUND OF THE INVENTION

Cancer is a serious threat to modern society. Worldwide, more than 10 million people are diagnosed with cancer every year and it is estimated that this number will grow to 15 million new cases every year by 2020. Cancer causes six million deaths every year or 12% of the deaths worldwide. Of the 1.2 million patients newly diagnosed with cancer in the United States annually, approximately 50% will be treated with radiation therapy as part of initial disease management. Approximately 150,000 additional patients with recurrent cancer may receive radiation therapy each year in the U.S. Chemotherapy is administered to about 350,000 cancer patients in the U.S. annually.

High levels of circulating cholesterol are associated with atherosclerosis, which may result in life-threatening blockages of blood vessels to the heart and brain. Unstable angina, myocardial infarction (heart attack) and sudden ischemic death remain the leading cause of morbidity and mortality in developed nations. Current estimates indicate that 1.1 million people in the U.S. annually will have a new or recurrent coronary attack, and over 45 percent of these patients may die from the coronary attack.

Texaphyrins are a class of molecules having a ring-shaped chemical structure and include both free base forms and the metallated forms (also known as metallated texaphyrins or texaphyrin metal complexes). The physical and chemical characteristics of texaphyrins are determined by the properties of the ring and the metal atom inserted into the ring. Texaphyrins selectively concentrate in diseased tissue such as tumor cells and atherosclerotic plaque inside blood vessels. Texaphyrins provide a valuable therapeutic approach to a broad range of diseases, including cancer, atherosclerosis and cardiovascular diseases.

### SUMMARY OF THE INVENTION

Described herein are methods, compositions, packaging systems, techniques and strategies for preserving and storing texaphyrins, including the free base forms and the metallated forms of texaphyrins, as well as packaged products of pharmaceutically-suitable solutions of texaphyrin metal complexes. Described herein are packaging systems for the prevention of oxygen degradation of pharmaceutical products such as texaphyrins, particularly the storage of texaphyrins for intravenous use. Also described herein are packaging systems, techniques and strategies for storing texaphyrin metal complexes, in both the powder form and in the solution form, for at least 6 months, for at least 12 months, for at least 18 months, for at least 24 months, for at least 30 months, or for at least 36 months, wherein the stored texaphyrin metal complexes degrade to produce less than 30 ppm of free metal. Also described herein are storage-stabilized packaged texaphyrin metal complexes, both in the powder form and in solution.

Disclosed herein are packaged products comprising high-purity texaphyrin metal complexes. Also disclosed herein are pharmaceutical packaged product compositions comprising such high-purity texaphyrin metal complexes. Also disclosed herein are methods for treating a disorder, disease or condition in a human patient comprising administering to the patient an effective amount of a texaphyrin metal complex that was stored as a storage-stabilized packaged product, both in the powder form and in solution.

One aspect disclosed herein is a storage-stabilized packaged product comprising an outer package opaque to visible light; at least one container inside the outer package, wherein the at least one container is (a) sealed with a seal that can be penetrated by a needle to gain access to the inside portion of the sealed container, (b) transparent to at least a portion of visible light, and (c) substantially impermeable to oxygen; and a solution inside the sealed container, wherein the solution comprises water and a compound having the structure of Formula (I):

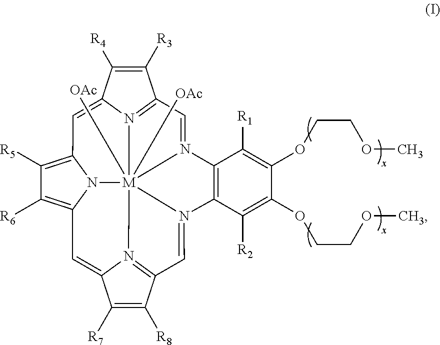

wherein M is a trivalent metal cation selected from the group consisting of Gd⁺³, and Lu⁺³; R₃, R₄, R₅, R₆, R₇ and R₈ are independently H, OH, CₙH₍₂ₙ₊₁₎O_{Y} or OCₙH₍₂ₙ₊₁₎O_{y} and R₁, R₂ are independently H or C₁-C₆ alkyl where at least one of R₃, R₄, R₅, R₆, R₇ and R₈ is CₙH₍₂ₙ₊₁₎O_{Y} or OCₙH₍₂ₙ₊₁₎O_{y}, having at least one hydroxy substituent; n is a positive integer from 1 to 11; y is zero or a positive integer less than or equal to n; each x is identical and is selected from the group consisting of 2, 3, 4, 5, and 6. In a further or alternative embodiment, the level of free M in the solution does not exceed about 30 ppm for at least about 1 year after the container is sealed.

In a further or alternative embodiment of the storage-stabilized packaged product of Formula (I), the outer packaging comprises a material selected from the group consisting of paper, paperboard, cardboard, metal foil, and plastic. In another embodiment of the packaged product of Formula (I), the outer packaging comprises paper, paperboard or cardboard. In yet another embodiment of the packaged product of Formula (I), the outer packaging is made of paper, paperboard or cardboard and the container is made of non-tinted borosilicate glass. In still yet another embodiment of the packaged product of Formula (I), the outer packaging is made of paper, paperboard or cardboard, the container is made of borosilicate glass and the seal comprises Teflon and butyl rubber. In yet another embodiment of the packaged product of Formula (I), the outer packaging is made of paper, paperboard or cardboard and M is Gd⁺³; R₄ and R₇ are C₃H₆OH; R₅ and R₆ are C₂H₅; R₃ and R₈ are CH₃; R₁ and R₂ are H; and x is 3.

In a further or alternative embodiment of the storage-stabilized packaged product of Formula (I), the container comprises material selected from high density polyethylene and glass. In another embodiment of the packaged product of Formula (I), the container is made of glass, including non-tinted glass. In yet another embodiment of the packaged product of Formula (I), the container is made of non-tinted glass and the seal comprises Teflon and butyl rubber. In yet another embodiment of the packaged product of Formula (I), the container is made of non-tinted borosilicate glass.

In a further or alternative embodiment of the packaged product of Formula (I), the seal comprises a material selected from Teflon and butyl rubber. In a further or alternative embodiment of the packaged product of Formula (I), the seal is made of Teflon and butyl rubber. In another embodiment of the packaged product of Formula (I), the seal is made of Teflon and butyl rubber and M is Gd⁺³; R₄ and R₇ are C₃H₆OH; R₅ and R₆ are C₂H₅; R₃ and R₈ are CH₃; R₁ and R₂ are H; and x is 3.

In a further or alternative embodiment of the packaged product of Formula (I), the solution partially fills the sealed container and the resulting head space is situated between the solution and the seal. In another embodiment of the packaged product of Formula (I), the solution partially fills the sealed container and the resulting head space is situated between the solution and the seal where the head space comprises at least about 90% nitrogen gas; at least about 95% nitrogen gas; occupies less than about 12% of the volume of the sealed container; occupies less than about 7% of the volume of the sealed container. In yet another embodiment of the packaged product of Formula (I), the solution partially fills the sealed container and the resulting head space is situated between the solution and the seal where the head space comprises at least about 90% nitrogen gas and M is Gd⁺³; R₄ and R₇ are C₃H₆OH; R₅ and R₆ are C₂H₅; R₃ and R₈ are CH₃; R₁ and R₂ are H; and x is 3. In still yet another embodiment of the packaged product of Formula (I), the solution partially fills the sealed container and the resulting head space is situated between the solution and the seal where the head space comprises at least about 90% nitrogen gas, or at least about 95% nitrogen gas, or occupies less than about 12% of the volume of the sealed container and the volume of solution in the sealed container is at least about 50 mL. In alternative embodiments, the volume of solution in the sealed container is at least about 100 mL, or at least 200 mL, or at least 500 mL. In still yet another embodiment of the packaged product of Formula (I), the head space is substantially deoxygenated prior to sealing the sealed container.

In a further or alternative embodiment of the packaged product of Formula (I), the solution further comprises an acid. In another embodiment of the packaged product of Formula (I), the acid is acetic acid. In yet another embodiment of the packaged product of Formula (I), the acid is acetic acid and the solution has a pH between about 4.5 and about 5.5; or between about 4.7 and 5.3. In still yet another embodiment of the packaged product of Formula (I), the acid is acetic acid and the solution has a pH between about 4.5 and about 5.5 and M is Gd⁺³; R₄ and R₇ are C₃H₆OH; R₅ and R₆ are C₂H₅; R₃ and R₈ are CH₃; R₁ and R₂ are H; and x is 3.

In a further or alternative embodiment of the packaged product of Formula (I), the solution further comprises an isotonic agent. In another embodiment of the packaged product of Formula (I), the solution further comprises an isotonic agent and M is Gd⁺³; R₄ and R₇ are C₃H₆OH; R₅ and R₆ are C₂H₅; R₃ and R₈ are CH₃; R₁ and R₂ are H; and x is 3. In a further or alternative embodiment of the packaged product of Formula (I), the isotonic agent is selected from the group consisting of saccharides, polyhydric alcohols, and dibasic sodium phosphate. In a further or alternative embodiment of the packaged product of Formula (I), the isotonic agent is a polyhydric alcohol selected from the group consisting of mannitol and sorbitol. In a further or alternative embodiment of the packaged product of Formula (I), the isotonic agent is about 3-10% mannitol; in another embodiment, about 4-6% mannitol.

In a further or alternative embodiment of the packaged product of Formula (I), the concentration of the compound of Formula (I) is between about 2.0 mg/mL and about 3.0 mg/mL; between about 2.2 mg/mL and about 2.8 mg/mL; between about 2.3 mg/mL and about 2.7 mg/mL; or between about 2.4 mg/mL and about 2.6 mg/mL. In yet another embodiment of the packaged product of Formula (I), the concentration of the compound of Formula (I) is between about 2.0 mg/mL and about 3.0 mg/mL and M is Gd⁺³; R₄ and R₇ are C₃H₆OH; R₅ and R₆ are C₂H₅; R₃ and R₈ are CH₃; R₁ and R₂ are H; and x is 3. In a further or alternative embodiment of the packaged product of Formula (I), the concentration of the compound of Formula (I) is about 2.5 mg/mL. In a further or alternative embodiment, the concentration of the compound of Formula (I) is about 2.0 mM, or about 2.2 mM, or about 2.4 mM.

In a further or alternative embodiment of the packaged product of Formula (I), M is Gd³⁺; R₄ and R₇ are C₃H₆OH; R₅ and R₆ are C₂H₅; R₃ and R₈ are CH₃; R₁ and R₂ are H; and x is 3. In another embodiment of the packaged product of Formula (I), M is Gd⁺³; R₄ and R₇ are C₃H₆OH; R₅ and R₆ are C₂H₅; R₃ and R₈ are CH₃; R₁ and R₂ are H; and x is 3 and the solution comprises less than about 30 ppm of Gd⁺³ not associated with the compound of Formula (I). In yet another embodiment of the packaged product of Formula (I), M is Gd⁺³; R₄ and R₇ are C₃H₆OH; R₅ and R₆ are C₂H₅; R₃ and R₈ are CH₃; R₁ and R₂ are H; and x is 3 where the solution comprises less than about 30 ppm of Gd⁺³ not associated with the compound of Formula (I), and the level of such free Gd⁺³ the solution does not exceed about 30 ppm for at least about 1 year; in another embodiment, for at least about 2 years; in another embodiment, for at least about 3 years.

In a further or alternative embodiment of the packaged product of Formula (I), the solution further comprises a buffer. In a further or alternative embodiment of the packaged product of Formula (I), the solution further comprises an anti-crystallizing agent. In a further or alternative embodiment of the packaged product of Formula (I), the solution further comprises a preservative. In a further or alternative embodiment of the packaged product of Formula (I), the solution does not contain an oxidizing agent other than the compound of Formula (I) and oxygen.

In a further embodiment of any of the aforementioned embodiments, the outer package contains a single sealed container. In another embodiment of any of the aforementioned embodiments, the packaged product is stored at a temperature between about 2-8° C. In a further embodiment of any of the aforementioned embodiments, the sealed container is substantially impermeable to water vapor. In a further embodiment of any of the aforementioned embodiments, the head space is substantially deoxygenated prior to sealing the sealed container.

In another embodiment of any of the aforementioned embodiments, the solution is suitable for administration to a human patient. In another embodiment of any of the aforementioned embodiments, the solution is suitable for intravenous administration to a human patient. In another embodiment of any of the aforementioned embodiments, the solution is suitable for intravenous administration. In another embodiment of any of the aforementioned embodiments, the patient is a human patient with cancer, or with a cardiovascular disease. In another embodiment, the cancer is selected from the group consisting of renal cell cancer, lymphoma, leukemia, lung cancer and brain metastases arising from lung cancer. In another embodiment of any of the aforementioned embodiments, the human patient has lung cancer that has metastasized to the brain of the patient. In another embodiment, the cardiovascular disease is selected from the group consisting of artherosclerosis, coronary artery disease, saphenous vein graft disease, and peripheral artery disease. In a further embodiment, M is Lu+3 and the subject is further provided photodynamic therapy. In one embodiment, the photodynamic therapy is provided after the subject has been administered the compound of Formula (I). In another embodiment, the target area is illuminated with light having wavelengths between about 725 and 760 nm.

Another aspect disclosed herein is a method for inhibiting the formation of free metal cation in a solution of a compound having the structure of Formula (I):

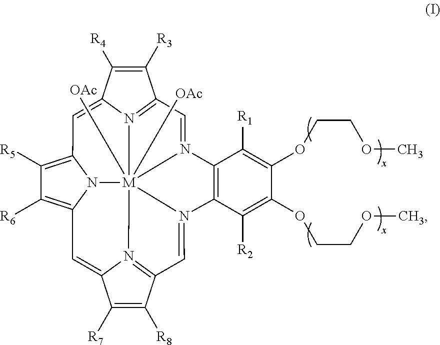

wherein M is a trivalent metal cation selected from the group consisting of Gd⁺³, and Lu⁺³; R₃, R₄, R₅, R₆, R₇ and R₈ are independently H, OH, CₙH₍₂ₙ₊₁₎O_{Y} or OCₙH₍₂ₙ₊₁₎O_{y} and R₁, R₂ are independently H or C₁-C₆ alkyl where at least one of R₃, R₄, R₅, R₆, R₇ and R₈ is CₙH₍₂ₙ₊₁₎O_{Y} or OCₙH₍₂ₙ₊₁)O_{y}, having at least one hydroxy substituent; n is a positive integer from 1 to 11; y is zero or a positive integer less than or equal to n; each x is identical and is selected from the group consisting of 2, 3, 4, 5, and 6, comprising using a storage-stabilized packaged product comprising an outer package opaque to visible light; at least one container inside the outer package, wherein the at least one container is (a) sealed with a seal that can be penetrated by a needle to gain access to the inside portion of the sealed container, (b) transparent to at least a portion of visible light, and (c) substantially impermeable to oxygen, wherein the solution of the compound having the structure of Formula (I) is within the sealed container. In a further or alternative embodiment, the level of free M in the solution does not exceed about 30 ppm for at least about 1 year after the container is sealed.

In a further or alternative embodiment of the method for inhibiting formation of free metal cation, the outer packaging comprises a material selected from the group consisting of paper, paperboard, cardboard, metal foil, and plastic. In another embodiment, the outer packaging comprises paper, paperboard or cardboard. In yet another embodiment, the outer packaging is made of paper, paperboard or cardboard and the container is made of non-tinted borosilicate glass. In still yet another embodiment, the outer packaging is made of paper, paperboard or cardboard, the container is made of borosilicate glass and the seal comprises Teflon and butyl rubber. In yet another embodiment, the outer packaging is made of paper, paperboard or cardboard and M is Gd⁺³; R₄ and R₇ are C₃H₆OH; R₅ and R₆ are C₂H₅; R₃ and R₈ are CH₃; R₁ and R₂ are H; and x is 3.

In a further or alternative embodiment of the method for inhibiting formation of free metal cation, the container comprises material selected from high density polyethylene and glass. In another embodiment, the container is made of glass, including non-tinted glass. In yet another embodiment, the container is made of non-tinted glass and the seal comprises Teflon and butyl rubber. In yet another embodiment, the container is made of non-tinted borosilicate glass.

In a further or alternative embodiment of the method for inhibiting formation of free metal cation, the seal comprises a material selected from Teflon and butyl rubber. In a further or alternative embodiment, the seal is made of Teflon and butyl rubber. In another embodiment, the seal is made of Teflon and butyl rubber and M is Gd+3; R4 and R7 are C3H₆OH; R5 and R6 are C₂H₅; R3 and R8 are CH3; R1 and R2 are H; and x is 3.

In a further or alternative embodiment, the solution partially fills the sealed container and the resulting head space is situated between the solution and the seal. In another embodiment, the solution partially fills the sealed container and the resulting head space is situated between the solution and the seal where the head space comprises at least about 90% nitrogen gas; at least about 95% nitrogen gas; occupies less than about 12% of the volume of the sealed container; occupies less than about 7% of the volume of the sealed container. In yet another embodiment, the solution partially fills the sealed container and the resulting head space is situated between the solution and the seal where the head space comprises at least about 90% nitrogen gas and M is Gd+3; R4 and R7 are C3H6OH; R5 and R6 are C₂H₅; R3 and R8 are CH3; R1 and R2 are H; and x is 3. In still yet another embodiment, the solution partially fills the sealed container and the resulting head space is situated between the solution and the seal where the head space comprises at least about 90% nitrogen gas, or at least about 95% nitrogen gas, or occupies less than about 12% of the volume of the sealed container and the volume of solution in the sealed container is at least about 50 mL. In alternative embodiments, the volume of solution in the sealed container is at least about 100 mL, or at least 200 mL, or at least 500 mL. In still yet another embodiment, the head space is substantially deoxygenated prior to sealing the sealed container.

In a further or alternative embodiment of the method for inhibiting formation of free metal cation, the solution further comprises an acid. In another embodiment, the acid is acetic acid. In yet another embodiment, the acid is acetic acid and the solution has a pH between about 4.5 and about 5.5; or between about 4.7 and 5.3. In still yet another embodiment, the acid is acetic acid and the solution has a pH between about 4.5 and about 5.5 and M is Gd+3; R4 and R7 are C3H6OH; R5 and R6 are C2H5; R3 and R8 are CH3; R1 and R2 are H; and x is 3.

In a further or alternative embodiment of the method for inhibiting formation of free metal cation, the solution further comprises an isotonic agent. In another embodiment, the solution further comprises an isotonic agent and M is Gd+3; R4 and R7 are C3H6OH; R5 and R6 are C2H5; R3 and R8 are CH3; R1 and R2 are H; and x is 3. In a further or alternative embodiment, the isotonic agent is selected from the group consisting of saccharides, polyhydric alcohols, and dibasic sodium phosphate. In a further or alternative embodiment, the isotonic agent is a polyhydric alcohol selected from the group consisting of mannitol and sorbitol. In a further or alternative embodiment, the isotonic agent is about 3-10% mannitol; in another embodiment, about 4-6% mannitol.

In a further or alternative embodiment of the method for inhibiting formation of free metal cation, the concentration of the compound of Formula (I) is between about 2.0 mg/mL and about 3.0 mg/mL. In a further or alternative embodiment of the packaged product of Formula (I), the concentration of the compound of Formula (I) is between about 2.0 mg/mL and about 3.0 mg/mL; between about 2.2 mg/mL and about 2.8 mg/mL; between about 2.3 mg/mL and about 2.7 mg/mL; or between about 2.4 mg/mL and about 2.6 mg/mL. In yet another embodiment, the concentration of the compound of Formula (I) is between about 2.0 mg/mL and about 3.0 mg/mL and M is Gd+3; R4 and R7 are C3H6OH; R5 and R6 are C₂H₅; R3 and R8 are CH3; R1 and R2 are H; and x is 3. In a further or alternative embodiment, the concentration of the compound of Formula (I) is about 2.5 mg/mL.

In a further or alternative embodiment of the method for inhibiting formation of free metal cation, M is Gd+3; R4 and R7 are C3H6OH; R5 and R6 are C₂H₅; R3 and R8 are CH3; R1 and R2 are H; and x is 3. In another embodiment, M is Gd+3; R4 and R7 are C3H6OH; R5 and R6 are C₂H₅; R3 and R8 are CH3; R1 and R2 are H; and x is 3 and the solution comprises less than about 30 ppm of Gd+3 not associated with the compound of Formula (I). In yet another embodiment, M is Gd+3; R4 and R7 are C3H6OH; R5 and R6 are C₂H₅; R3 and R8 are CH3; R1 and R2 are H; and x is 3 where the solution comprises less than about 30 ppm of Gd+3 not associated with the compound of Formula (I), and the level of such free Gd+3 in the solution does not exceed about 30 ppm for at least about 1 year; in another embodiment, for at least about 2 years; in another embodiment, for at least about 3 years.

In a further or alternative embodiment of the method for inhibiting formation of free metal cation, the solution further comprises a buffer. In a further or alternative embodiment, the solution further comprises an anti-crystallizing agent. In a further or alternative embodiment, the solution further comprises a preservative. In a further or alternative embodiment, the solution does not contain an oxidizing agent other than the compound of Formula (I) and oxygen.

In a further embodiment of any of the aforementioned embodiments, the outer package contains a single sealed container. In another embodiment of any of the aforementioned embodiments, the packaged product is stored at a temperature between about 2-8° C. In a further embodiment of any of the aforementioned embodiments, the sealed container is substantially impermeable to water vapor. In a further embodiment of any of the aforementioned embodiments, the head space is substantially deoxygenated prior to sealing the sealed container.

In another embodiment of any of the aforementioned embodiments, the solution is suitable for administration to a human patient. In another embodiment of any of the aforementioned embodiments, the solution is suitable for intravenous administration to a human patient. In another embodiment of any of the aforementioned embodiments, the solution is suitable for intravenous administration. In another embodiment of any of the aforementioned embodiments, the patient is a human patient with cancer, or with atherosclerosis. In another embodiment, the cancer is selected from the group consisting of renal cell cancer, lymphoma, leukemia, lung cancer and brain metastases arising from lung cancer. In another embodiment of any of the aforementioned embodiments, the human patient has lung cancer that has metastasized to the brain of the patient.

Another aspect described herein are packaged products comprising an outer packaging opaque to visible light; at least one container with a seal, wherein the at least one sealed container is (a) within the outer packaging, (b) transparent to at least a portion of visible light, and (c) substantially impermeable to oxygen; and a powder inside the sealed container, wherein the powder comprises a compound having the structure of Formula (I):

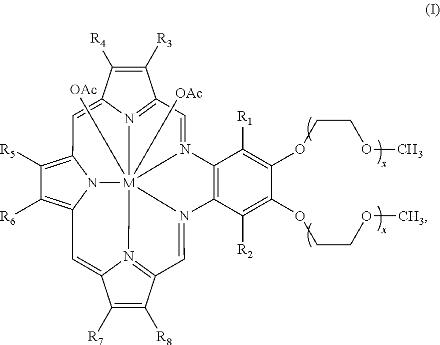

wherein M is a trivalent metal cation selected from the group consisting of Gd⁺³, and Lu⁺³; R₃, R₄, R₅, R₆, R₇ and R₈ are independently H, OH, CₙH_{(2n+1)O}_{Y} or OCₙH_{(2n+1)O}_{y} and R₁, R₂ are independently H or C₁-C₆ alkyl where at least one of R₃, R₄, R₅, R₆, R₇ and R₈ is CₙH₍₂ₙ₊₁₎O_{Y} or OCₙH_{(2n+1)O}_{y}, having at least one hydroxy substituent; n is a positive integer from 1 to 11; y is zero or a positive integer less than or equal to n; each x is identical and is selected from the group consisting of 2, 3, 4, 5, and 6. In a further or alternative embodiment, the level of free M in the solution does not exceed about 30 ppm for at least about 1 year after the container is sealed.

In a further or alternative embodiment, the outer packaging comprises a material selected from the group consisting of paper, paperboard, cardboard, metal foil, and plastic. In a further or alternative embodiment of the second aspect of Formula (I), the outer packaging comprises paper, paperboard, or cardboard.

In a further or alternative embodiment, the container comprises a material selected from high density polyethylene and glass. In a further or alternative embodiment of the second aspect of Formula (I), the container comprises glass. In a further or alternative embodiment of the second aspect of Formula (I), the container comprises non-tinted glass. In a further or alternative embodiment of the second aspect of Formula (I), the container comprises borosilicate glass. In a further or alternative embodiment of the second aspect of Formula (I), the container comprises non-tinted borosilicate glass.

In a further or alternative embodiment, the seal comprises a material selected from Teflon and butyl rubber. In a further or alternative embodiment, the seal comprises Teflon and butyl rubber. In a further or alternative embodiment, the seal can be penetrated by a needled to gain access to the interior of the sealed container. In a further or alternative embodiment, the powder can be dissolved in a solution comprising water and acetic acid to form a pharmaceutically acceptable solution. In a further or alternative embodiment, the pharmaceutically acceptable solution is suitable for intravenous administration to a patient. In a further or alternative embodiment the patient is a human patient with cancer, or with atherosclerosis. In another embodiment, the cancer is selected from the group consisting of renal cell cancer, lymphoma, leukemia, lung cancer and brain metastases arising from lung cancer. In a further or alternative embodiment the human patient has lung cancer that has metastasized to the brain of the patient.

In a further or alternative embodiment, M is Gd+3; R4 and R7 are C3H6OH; R5 and R6 are C2H5; R3 and R8 are CH3; R1 and R2 are H; and x is 3.

In a further or alternative embodiment, the sealed container further comprises a preservative. In a further or alternative embodiment, the sealed container does not contain an oxidizing agent other than the compound of Formula (I) and oxygen.

In a further embodiment of any of the aforementioned embodiments, the outer package contains a single sealed container. In another embodiment of any of the aforementioned embodiments, the packaged product is stored at a temperature between about 2-8° C. In a further embodiment of any of the aforementioned embodiments, the sealed container is substantially impermeable to water vapor. In a further embodiment of any of the aforementioned embodiments, the head space is substantially deoxygenated prior to sealing the sealed container.

In a further or alternative embodiment, the powder is suitable for reconstitution into a solution for administration to a human patient. In a further or alternative embodiment, the powder is suitable for reconstitution into a solution for intravenous administration to a human patient. In a further or alternative embodiment, the powder is suitable for reconstitution into a solution for intravenous administration to a human patient with cancer. In another embodiment of any of the aforementioned embodiments, the patient is a human patient with cancer, or with atherosclerosis. In another embodiment, the cancer is selected from the group consisting of renal cell cancer, lymphoma, leukemia, lung cancer and brain metastases arising from lung cancer. In another embodiment of any of the aforementioned embodiments, the human patient has lung cancer that has metastasized to the brain of the patient.

Another aspect disclosed herein are packaging systems for inhibiting the formation of free metal cation in a solution of a compound having the structure of Formula (I):

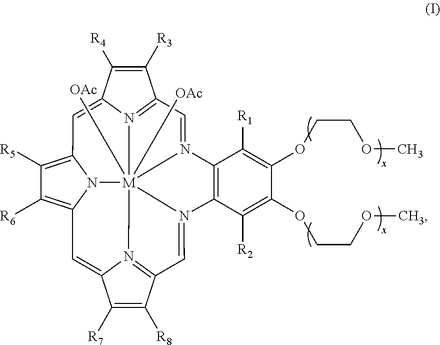

wherein M is a trivalent metal cation selected from the group consisting of Gd⁺³, and Lu⁺³; R₃, R₄, R₅, R₆, R₇ and R₈ are independently H, OH, CₙH_{(2n+1)O}_{Y} or OCₙH₍₂ₙ₊₁₎O_{y} and R₁, R₂ are independently H or C₁-C₆ alkyl where at least one of R₃, R₄, R₅, R₆, R₇ and R₈ is CₙH₍₂ₙ₊₁₎O_{Y} or OCₙH₍₂ₙ₊₁₎O_{y}, having at least one hydroxy substituent; n is a positive integer from 1 to 11; y is zero or a positive integer less than or equal to n; each x is identical and is selected from the group consisting of 2, 3, 4, 5, and 6, comprising an outer package opaque to visible light; at least one container inside the outer package, wherein the at least one container is (a) sealed with a seal that can be penetrated by a needle to gain access to the inside portion of the sealed container, (b) transparent to at least a portion of visible light, and (c) substantially impermeable to oxygen, wherein the solution of the compound having the structure of Formula (I) is within the sealed container. In a further or alternative embodiment, the level of free M in the solution does not exceed about 30 ppm for at least about 1 year after the container is sealed.

Another aspect disclosed herein are aqueous solutions of a compound having the structure of Formula (I):

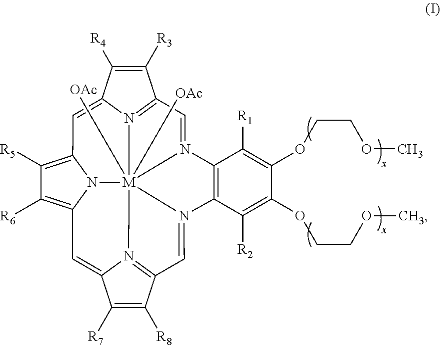

wherein M is a trivalent metal cation selected from the group consisting of Gd⁺³, and Lu⁺³; R₃, R₄, R₅, R₆, R₇ and R₈ are independently H, OH, CₙH₍₂ₙ₊₁₎O_{Y} or OCₙH₍₂ₙ₊₁₎O_{y} and R₁, R₂ are independently H or C₁-C₆ alkyl where at least one of R₃, R₄, R₅, R₆, R₇ and R₈ is CₙH₍₂ₙ₊₁₎O_{Y} or OCₙH₍₂ₙ₊₁₎, having at least one hydroxy substituent; n is a positive integer from 1 to 11; y is zero or a positive integer less than or equal to n; each x is identical and is selected from the group consisting of 2, 3, 4, 5, and 6, wherein the solution has been stored for at least 6 months in a packaging system comprising an outer package opaque to visible light; at least one container inside the outer package, wherein the at least one container is (a) sealed with a seal that can be penetrated by a needle to gain access to the inside portion of the sealed container, (b) transparent to at least a portion of visible light, and (c) substantially impermeable to oxygen, wherein the solution of the compound having the structure of Formula (I) is within the sealed container, and wherein the level of free M in the solution does not exceed about 30 ppm. In a further embodiment, the solution has been stored for at least 12 months; in another embodiment, at least 18 months; in another embodiment, at least 24 months; in another embodiment, at least 30 months; and in another embodiment, at least 36 months. In a further embodiment of any of the aforementioned embodiments, the outer package contains a single sealed container. In another embodiment of any of the aforementioned embodiments, the packaged product is stored at a temperature between about 2-8° C. In a further embodiment of any of the aforementioned embodiments, the sealed container is substantially impermeable to water vapor. In a further embodiment of any of the aforementioned embodiments, the head space is substantially deoxygenated prior to sealing the sealed container. In another embodiment of any of the aforementioned embodiments, the solution is suitable for administration to a human patient. In another embodiment of any of the aforementioned embodiments, the solution is suitable for intravenous administration to a human patient. In another embodiment of any of the aforementioned embodiments, the solution is suitable for intravenous administration. In another embodiment of any of the aforementioned embodiments, the patient is a human patient with cancer, or with atherosclerosis. In another embodiment, the cancer is selected from the group consisting of renal cell cancer, lymphoma, leukemia, lung cancer and brain metastases arising from lung cancer. In another embodiment of any of the aforementioned embodiments, the human patient has lung cancer that has metastasized to the brain of the patient.

As used herein and in the appended claims, the singular forms “a,” “an,” and “the” include plural reference unless the context clearly indicates otherwise.

As used herein, the term “substantially impermeable to oxygen” refers to the permeability of the packaging material to oxygen. A packaging material that is substantially impermeable to oxygen limits the amount of oxygen entering the interior of the packaging material. In certain embodiments, the per annum increase in the level of oxygen is less than about 5%, 4%, 3%, 2%, 1% or 0.5%. The less oxygen that passes through the packaging material per annum, the less the amount of metallated texaphyrin that decomposes per annum.

The term “substantially deoxygenated,” as used herein, refers to a product that has been packaged whereby the packaged environment has been reduced in oxygen concentration. In certain embodiments, the oxygen concentration is less than about 100000 ppm, the oxygen concentration is less than about 50000 ppm, the oxygen concentration is less than about 10000 ppm, the oxygen concentration is less than about 5000 ppm, the oxygen concentration is less than about 3000 ppm, the oxygen concentration is less than about 1000 ppm, the oxygen concentration is less than about 100 ppm, the oxygen concentration is less than about 50 ppm, or the oxygen concentration is less than about 20 ppm.

As used herein, the term “head space” refers to the region between the solution or powder and the container. Head space can also be referred to as the portion of the container that does not contain the solution or powder. By way of example, the head space may contain air, nitrogen gas, or similar such elements.

As used herein, the term “buffer” refers to a substance that minimizes change in the pH of a solution.

As used herein, the term “anti-crystallizing agent” refers to a substance that reduces the formation of crystals within the solution.

As used herein, the term “preservative” refers to a substance that reduces spoilage.

As used herein, the term “degradation” refers to any change to a drug that may occur during storage resulting in (a) an undesirable by-product, for example by hydrolysis or oxidation of the drug, or undesirable form, such as crystals, or (b) loss of the drug, for example through absorption into other materials within the packaging, or evaporation. In one embodiment, degradation refers to loss of the metal atom from a metallated texaphyrin, including the loss of Gd3+ or Lu3+ from gadolinium-metallated and lutetium-metallated texaphyrin complexes, respectively.

The term “photodynamic therapy” as described herein, refers to a treatment that combines a light source and a photosensitizing agent (a drug that is activated by light). As used herein, the term “substantially impermeable to water vapor” means that the product package has a moisture vapor transmission rate not greater than about 0.0002 g/day/in. sq. at 40° C./75% Relative Humidity (RH). Therefore, for a typical shelf life of two years, the product package should permit not greater than about 1 g of moisture to pass through at 25° C./60% RH.

It is to be understood that the methods, compositions, packaging systems, techniques and strategies for preserving and storing texaphyrins and texaphyrin metal complexes, as well as packaged products of texaphyrin metal complexes that are described herein are not limited to the particular methodology, protocols, environments, systems, and reagents described herein and as such may vary. It is also to be understood that the terminology used herein is for the purpose of describing particular embodiments only, and is not intended to limit the scope of the methods, compositions, packaging systems, techniques and strategies for preserving and storing texaphyrins and texaphyrin metal complexes, as well as packaged products of texaphyrin metal complexes described herein, which will be limited only by the appended claims as interpreted in light of the specification.

### BRIEF DESCRIPTION OF THE FIGURES

A better understanding of the features and advantages of the methods, compositions, packaging systems, techniques and strategies for preserving and storing texaphyrins and texaphyrin metal complexes, as well as packaged products of texaphyrin metal complexes that are described herein may be obtained by reference to the following detailed description that sets forth illustrative embodiments, and the accompanying drawings of which:

FIG. 1 presents a flow chart representation of the relationship of certain aspects of the packaged product described herein.

FIG. 2 presents an illustrative, non-limiting example of a packaged bottle product.

FIG. 3 presents an illustrative, non-limiting example of a packaged syringe product.

FIG. 4 presents an illustrative, non-limiting example of bottle with minimal exposed surface area and head space.

### DETAILED DESCRIPTION OF THE INVENTION

### Texaphyrins

Sessler et at reported in U.S. Pat. No. 5,994,535, incorporated herein by reference in its entirety, the synthesis of “texaphyrins,” which absorb strongly in the tissue-transparent 730-770 nm range. Such compounds are capable of existing in both the free-base form and of supporting the formation of complexes with a variety of metal cations, such as Cd²⁺, Hg²⁺, In³⁺, Y³⁺, Nd³⁺, Eu³⁺, Sm³⁺, La³⁺, Lu³⁺, Gd³⁺, and other cations of the lanthanide series. Texaphyrins find use in the treatment of cancer and atherosclerosis.

Metallated texaphyrins, upon exposure to an oxidizing agent, such as oxygen, can decompose and lose the coordinated metal atom. Certain frequencies of light (including those transmitted through amber glass) can assist in the decomposition of metallated texaphyrins. Such decomposition is slow, and indeed negligible over the time frame associated with the actual use and administration of metallated texaphyrins for therapeutic and/or diagnostic purposes. However, upon extended storage, the amount of decomposition product can accumulate to unacceptable levels, including, by way of example only greater than about 30 ppm of free Gd3+ in the case of motexafin gadolinium.

The extended storage of metallated texaphyrins is an important aspect of their use as therapeutic and/or diagnostic agents. To be practicable, the shelf-life (i.e., the time during which the level of decomposition products is maintained at a safe level and during which time the effectiveness of the therapeutic agent is maintained) of metallated texaphyrins should be at least 6 months, more preferably 12 months, even more preferably 18 months, still more preferably 24 months, still more preferably 30 months, and even more preferably 36 months. As such, the hospital or physician can place an order for the metallated texaphyrin, safely store the agent, and us the agent when needed, without having to worry about an inconveniently foreshortened shelf life. As such, methods, strategies, compositions, packaging systems and packaging techniques for extending the effective shelf life of metallated texaphyrins provide significant benefit.

There are a number of disadvantages associated with a low purity sample of texaphyrins, including, by way of example only, difficulties in solubility optimization of the pharmaceutical formulations such that the effective concentrations of the texaphyrins may vary from preparation to preparation; difficulties in optimizing drug dosage; and difficulties in stabilizing formulations. The improvement in the purity of the packaged compounds disclosed herein overcomes such disadvantages of solubility, dose optimization, and drug stabilization. The improvement in the purity further results in a decrease in side effects associated with low purity pharmaceutical formulations, an improvement in the measured half life of the drug formulation, and an improvement in drug targeting.

### Packaged Product

### Introduction

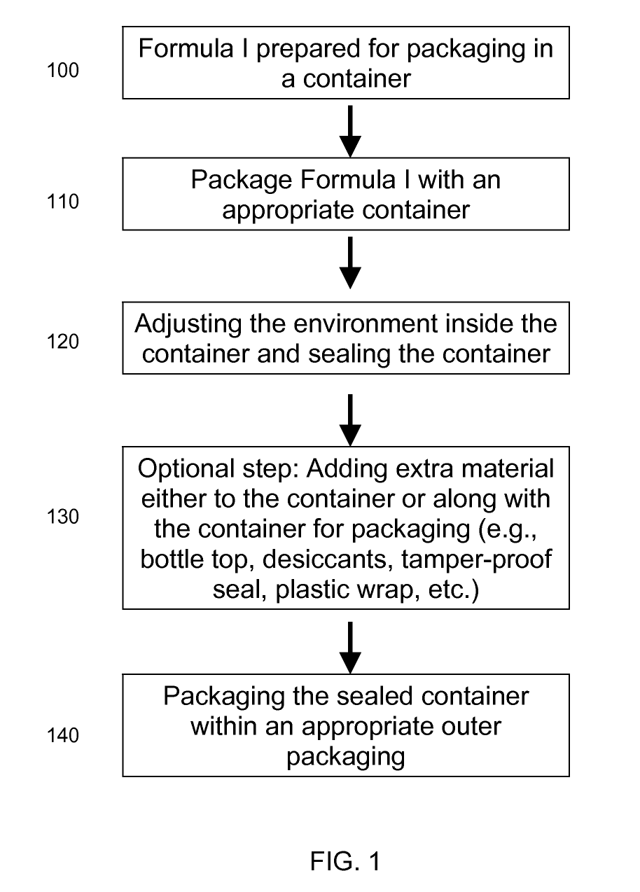

FIG. 1 depicts a flow chart of one embodiment of packaging Formula (I). Formula (I) may be prepared for packaging 100 in different forms such as a solution or powder, for example. Depending on the form of Formula (I), an appropriate container 110 suitable to hold Formula (I) may be used. Also dependent upon the container chosen, 120 sealing the container and adjusting the environment inside the container for packing will be done. Optional steps 130 may involve adding extra materials either to the container or along with the container for packaging, by way of example only includes a bottle top, desiccants, tamper-proof seal, plastic wrap and the like. Finally the 140 sealed container containing Formula (I) is packaged within an appropriate outer package.

### Outer Packaging: Types of Outer Packaging:

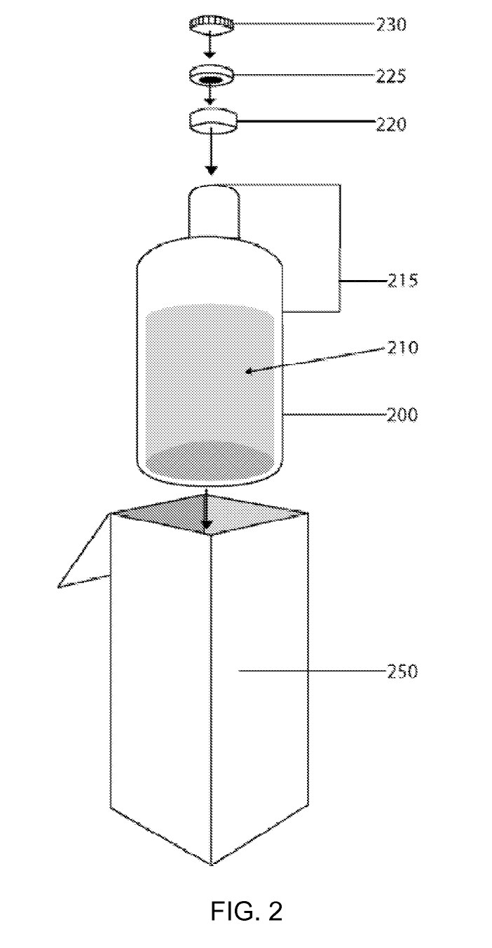

FIG. 2 depicts a vial or container 200 with a seal 220, containing Formula (I) 210, that fits into an outer packaging 250. In this depicted embodiment, the outer packaging 250 is a paper box. In one embodiment, the outer packaging 250 protects the container 200 with seal 220 and contents (a solution of Formula (I)) 210 from light. In further or alternative embodiments, the outer packaging 250 protects the container 200 with seal 220 along with an aluminum seal protector 225 and its contents of Formula (I) 210 from sunlight, ultraviolet light, contaminants, degradation, impurities, other solutions and spillage. The outer packaging 250 will not significantly absorb, react with, or otherwise adversely affect the Formula (I) drug 210 or other excipients or components used in intravenous delivery during storage of the drug prior to its use. The outer packaging may be in any shape or form which protects container 200 with seal 220 and its contents of Formula (I) 210, including, by way of example only a paper box 250, a cardboard box, a carton, a plastic bag, a fabric case, a metal receptacle, a wooden bin or the like.

### Qualification Standards for Packaging

The qualification standards for packaging a vial or sealed container within an outer packaging depends on the type of vial or sealed container and/or the type of outer packaging 140, see FIG. 1. In one embodiment, a combination of a sealed glass vial and paper box is used as protection material for packaging of Formula (I), see FIG. 2. Such a combination provides maximum protection from light degradation as well as oxygen degradation. Such varied combinations of packaging also provide a protective environment for solutions containing Formula (I) from outside temperatures ranging from about 0-3° C., about 2.5-4.5° C., about 3.5-5.5° C., about 4.5-7.5° C. and about 5.0-8.5° C. In one embodiment, these storage-stabilized packaged formulations are stored at room temperature or in a standard refrigerator or at temperatures from about 2 to 8° C. or about 2 to 5° C.

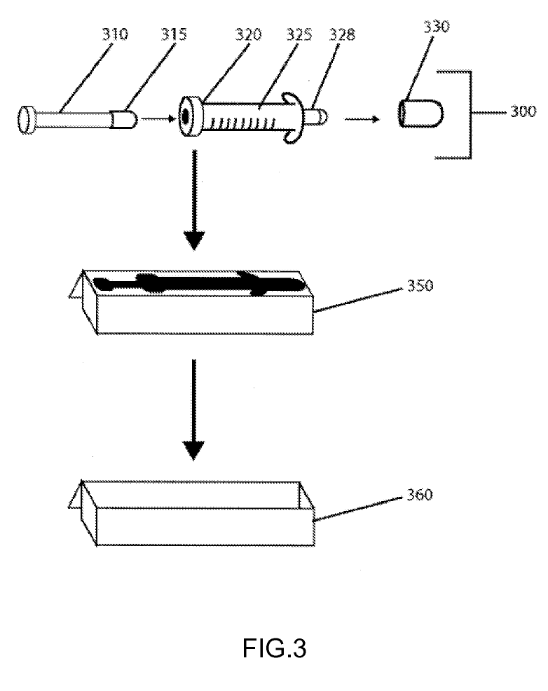

Further or alternative embodiments, by way of example only, include a combination of a syringe sealed in plastic with a cardboard box, a combination of a syringe sealed in plastic with an outer nontransparent paper lining, a combination of a glass bottle sealed in plastic with a cardboard box, a combination of a plastic bottle with a cardboard box, a combination of a plastic bottle sealed in plastic with a cardboard box, a combination of a glass bottle encased in a Styrofoam case within a cardboard box, a combination of a syringe 300 encased in a Styrofoam case 350 within a cardboard box 360, see FIG. 3, and the like. The qualification standards for other such combinations of sealed containers and outer packaging differ because of the different materials used in the container and outer packaging. However, any combination should provide protection from contamination, such as the crystallization or degradation, of the drug, and from other environmental factors, during storage of the system prior to its use.

In further or alternative embodiments a desiccant or other such materials which are substantially absorbent to water vapor may accompany the vial and or sealed container within the outer packaging 130, see FIG. 1. Suitable materials for use as desiccants include oxides of aluminum, calcium, titanium, zirconium, silicon, thorium, magnesium and barium, alumina, alumina hydrates, natural and synthetic molecular sieves, silica gel, precipitated silica, clays, perchlorates, zeolite, natural gums, magnesium or calcium sulfate, calcium, lithium or cobalt chloride, and calcium carbonate. An indicator dye can also be added to the desiccant material to provide for monitoring the amount of moisture absorbed during storage of the product package.

The amount of desiccant that may used will depend on several factors including the moisture permeability of the types of materials used in making the product package, the moisture absorbing capacity of the particular desiccant material, and the intended shelf life of the drug. The minimal amount to be used is that amount that will effectively absorb water vapor within the product package over the intended shelf life of the drug, typically three years, and achieve an acceptable level of drug loss from crystallization or degradation to still deliver a therapeutically effective amount of the drug. The desiccant should be capable of absorbing at least about 1.5 grams to about 5 grams of moisture over the intended period of storage and use of the product package. The amount of desiccant material needed to prevent such moisture contamination can be determined by one skilled in the art through routine experimentation.

The suitable desiccant material may be incorporated into the product package in any manner including a compressed pellet, or enclosed within a holder such as a capsule, sachet or container. Any material that is water vapor permeable and does not react with or adversely affect (for example, by leaching or absorption) components of the formulation of Formula (I) or other materials used in making the product package is suitable for forming the desiccant holder. Such materials include polyethylene, polyethylene terephthalate, polypropylene, coated and non-coated paper, and perforated sheet and laminate materials. In one embodiment, the material for the desiccant holder may be non-woven polyolefin.

In further or alternative embodiments, the outer packaging contains oxygen-absorbing materials, in particular, when the outer packaging comprises a sealed container, such as a plastic bag or bottle. The oxygen-absorbing material can provide further protection to the solution Formula (I) formulations by absorbing keeping the level of oxygen surrounding the sealed container (i.e., the container which directly contains the Formula (I) formulation) to a minimum. Examples of oxygen-absorbing materials (also known as oxygen scavengers) include ascorbic acid, iron powder, and an inorganic ferrous salt such as ferrous-halide, -nitrate or -sulfide. Such oxygen-absorbing materials can be kept within an oxygen-permeable container, such as a sachet.

Container with a Seal

### Types of Containers

FIG. 2 depicts a vial or container 200 with a seal 220, containing Formula (I) 210, that fits into an outer packaging 250. The container aids to protect its contents of Formula (I) from contaminants, degradation, impurities, other solutions and/or spillage 110, see FIG. 1. The container forms a protective environment to house Formula (I) so as to slow the effects of degradation of Formula (I). Further alternative embodiments of different container types include, by way of example only, a high density polyethylene container, a plastic bottle, a syringe, a “drip bag,” a pre-filled syringe, an intravenous bag, and the like. In further or alternative embodiments, the container contains a compound of Formula (I) in solution—including a concentrated solution that can be diluted down to a desired concentration or at a concentration ready for administration to the patient. Alternatively, the container can contain a solid dosage form of a compound of Formula (I), wherein the solid dosage form can be dissolved in an appropriate solution to create a formulation having a desired concentration. The solid dosage form can include a powder, including a lyophilized powder; semi-crystalline material; crystalline material; grains; granules and the like. Alternatively, the container can contain a semi-solid dosage form of a compound of Formula (I), including a gel or jelly, wherein the semi-solid dosage form can be dissolved in an appropriate solution to create a formulation having a desired concentration. Thus, in any of the container embodiments described herein, the compound of Formula (I) can be in the form of a solid, semi-solid, or solution, and further may be either ready to use (i.e., administer to a patient), or available for formulation to a desired pharmaceutical dosage form, including an intravenously-acceptable formulation.

FIG. 3 shows Formula (I) 325 within the barrel 320 of a syringe as well as the components of the syringe: plunger 310, rubber component 315, tip 328, and tip protector 330. The container not only stores Formula (I) for transportation to a human subject for intravenous use of the drug, but also prevents degradation of Formula (I) by shielding it from the corrosive effects of light and oxygen. Prior to sealing of Formula (I) in a container, the container is gas flushed with nitrogen gas and/or evacuated of oxygen. In further embodiments, other non-oxygen gases are used instead of nitrogen, including by way of example only, argon and neon, or mixtures of such gases. In one embodiment, the container will have about less than 10% oxygen or about less than 5% oxygen. The container will not significantly absorb, react with, or otherwise adversely affect the Formula (I) drug or other excipients or components used in the intravenous delivery system during storage of the drug prior to its use.

### Types of Seals

FIG. 2 depicts a vial 200, containing Formula (I) 210, a seal 220 and a seal protector 225 that is placed at the opening of vial 200. In one embodiment, the seal 220 aids to protect the vial's 200 contents of Formula (I) 210 from contaminants, degradation, impurities, other solutions and spillage. The seal 220 encloses the protective environment of the container 200 to house Formula (I) 210 so as to slow the effects of degradation of Formula (I) 210. In one embodiment, the seal is a one piece elastomeric bottle stopper which forms a tight seal onto a glass bottle container housing Formula (I). This seal can be penetrated by a needle to gain access to the inside portion of the sealed container. Further alternative embodiments of seals include, by way of example only, a syringe plunger's rubber component 315, a syringe tip protector 330, an airtight reinforced plastic seal for a bottle and the like.

### Qualification Standards for Containers

The qualification standards for a vial or sealed container varies depending on the type of vial or sealed container used and which form of Formula (I) is used. By way of example only, a sealed syringe housing a powder form of Formula (I) or a sealed bottle housing a powder form of Formula (I) may withstand higher temperatures than a sealed syringe housing a liquid form of Formula (I) or a sealed bottle housing a liquid form of Formula (I) which may lead to a higher rate of degradation of the drug. In one embodiment, the container housing the drug is in an oxygen depleted environment which is sealed and substantially airtight. However, any combination should provide protection from contamination, such as the crystallization or degradation, of the drug, and from other environmental factors, during storage of the system prior to its use.

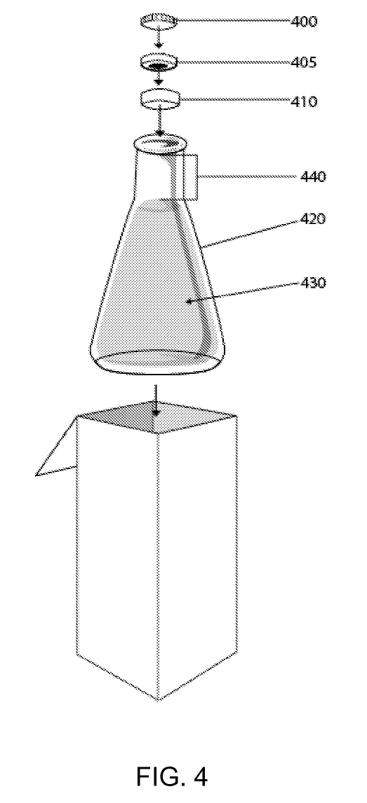

In another embodiment, the liquid form of Formula (I) is housed in a container with a minimal amount of headspace for storage 120, see FIG. 1, FIG. 2, 215 and FIG. 4, 440. The headspace may contain at least about 90% nitrogen gas, or at least about 95% nitrogen gas and occupy either less than about 12% or less than about 7% of the volume of the sealed container. In still a further embodiment, the liquid form of Formula (I) is flushed with nitrogen inside the container. In a further embodiment, a non-oxygen gas (including nitrogen, argon, neon or combinations thereof) is flushed into the empty container followed by the solution of Formula (I); alternatively, the solution of Formula (I) partially fills the container and the remaining head space is flushed with a non-oxygen gas. In another example, FIG. 4 depicts a triangular shaped bottle 420 would provide a small exposed surface area 440 for the liquid form of Formula (I) 430 to be in contact with the air inside the container. This may decrease the degradation of Formula (I) 430 by providing less surface area 440 for the liquid form to be in contact with potentially corrosive substances.

In further or alternative embodiments, a protective cap may accompany the bottle seal or syringe tip seal 130, see FIG. 1. The protective cap may prevent unintentional damage to the bottle or syringe tip seal before use. In another embodiment, the protective cap may be child-resistant to prevent unintentional opening by a minor before use 130, see FIG. 1. In still further or alternative embodiments, a plastic bag, a foil wrapped container or other such materials may seal the vial and/or sealed container within the outer packaging. The plastic bag or foil wrapped container may provide another protective layer against light, contaminants, degradation, impurities, other solutions and spillage 130, see FIG. 1.

In order to effectively achieve the mechanical strength characteristics to be called “child-resistant” (i.e., substantially impairs a child's ability to open a container with its hands as determined in accordance with the procedures set forth in the Requirements for the Special Packaging of Household Substances with changes cited in the Federal Register, Vol. 60, No. 140, pp. 37710-3744, 1995), a bi-layered structure for the inner pouch (which is placed around the sealed container containing the Formula (I) formulation and the outer packaging) may be used, however, a single film or sheet with an appropriate thickness or tear resistance could be employed.

Formula (I) Product: Forms of Formula (I) that may be Packaged

One embodiment described herein is a packaged product of Formula (I) for intravenous drug use to a human subject wherein the packaging will not significantly absorb, react with, or otherwise adversely affect the drug or other excipients or components used in intravenous delivery during storage of the system prior to its use. In a further embodiment described herein are packaged products of Formula (I) for intravenous delivery, comprising a high-purity texaphyrin metal complex of Formula (I). The foregoing and other objectives are achieved by providing light protective materials and a substantially deoxygenated environment to prevent degradation to Formula (I) prior to use. Such light protective materials include an outer packaging that is opaque and an inner package that comprises a transparent, non-tinted material, such as glass. The packaging of Formula (I) for intravenous use is dependent on the form of the drug 100, see FIG. 1. In one embodiment, Formula (I) may be packaged in liquid form. In another embodiment, Formula (I) may be packaged in powder form with reconstituting solution.

Suitable storage-stabilized formulations of Formula (I) include a solution of Formula (I) in water and acetic acid. In one embodiment, the storage-stabilized formulation should have a pH of 5.4. In other embodiments, the storage-stabilized formulation should have a pH between about 4.5-5.5, about 5.0-5.9 or about 4.9-5.9. In another embodiment the concentration of Formula (I) in the storage-stabilized formulation is between 2.5 mg/mL and about 3.0 mg/mL; in a further embodiment the concentration of Formula (I) is about 2.5 mg/mL.

In further or alternative embodiments, storage-stabilized formulation contains an isotonic agent, which can include electrolytes and/or non-electrolytes. Non-limiting examples of electrolytes includes sodium chloride, potassium chloride, dibasic sodium phosphate, sodium gluconate and combinations thereof. Non-limiting examples of non-electrolytes includes saccharides and polyhydric alcohols; further examples include mannitol, sorbitol, glucose, dextrose, glycerol, xylitol, fructose, maltose, mannose, glycerin, propylene glycol, and combinations thereof. In still further embodiments, the storage-stabilized formulation comprises a buffer, an anti-crystallizing agent, and/or a preservative. Buffering agents aid in stabilizing pH. Anti-crystallizing agents aid in stabilizing the concentration of the solution. Preservatives aid in preventing the growth of micro-organisms, and include by way of example only, methyl paraben, propyl paraben, benzyl alcohol, sodium hypochlorite, phenoxy ethanol and/or propylene glycol. In one, the storage-stabilized formulation does not contain an oxidizing agent other than Formula (I) and oxygen. Oxidizing agents promote degradation of the compound of Formula (I).

### General Packaging Specifications

The packaging system may be prepared by loading the product package contents (i.e., Formula (I), bottle, syringe, plastic bag, desiccant, cardboard box) by means of any suitable or conventional manufacturing operation and sealing process. The sealing process may include gas flushing or evacuation of oxygen from the container.

The degradation of solutions comprising Formula (I) can be measured by the levels of free metal ion, including Gd⁺³ and Lu³⁺. In one embodiment, accumulation of less than 30 ppm of free metal ion, including Gd+3, within the packaged product is desired. In another embodiment, the accumulation of free metal ion, including Gd+3, within the packaged product should not exceed 30 ppm for at least about 1 year. In yet another embodiment, the accumulation of free metal ion, including Gd+3, within the packaged product should not exceed 30 ppm for at least about 3 years. Whether Formula (I) is packaged as a solution or powder form for reconstitution before use, measurement of Gd+3 levels can be an indication of degradation or spoilage.

In one embodiment, Formula (I) may be packaged in powder form with reconstituting solution. Reconstitution is achieved by admixing the Formula (I) powder with a solution comprising, e.g., water, acetic acid and mannitol, using amounts and concentrations as described for the Formula (I) solutions described herein. The term “powder” is used to generically describe any solid form of Formula (I) in a particulate form, including crystalline forms and non-crystalline forms, or grains, beads, chunks, fine powders, coarse powder or other particulate forms.

### Illustrative Methods for Determining Purity of a Sample or Composition

Without limiting the scope of the compositions, methods and packaged products disclosed herein, some of the methods for demonstrating the purity of the pharmaceutical formulations are described below. Such methods can be used to monitor or demonstrate the suitability of a particular package system for storing a pharmaceutical formulation over time, including for at least 6 months, 1 year, 18 months, 2 years, 30 months, and 3 years, as desired.

Chromatographic methods that are contemplated to be used for demonstrating or monitoring purity are, by way of example only, molecular size exclusion chromatography, native gel electrophoresis, high pressure liquid chromatography (HPLC), liquid chromatography (LC), gas chromatography (GC), GC coupled with mass spectroscopy (GC MS), supercritical fluid chromatography, gel permeation chromatography and ion exchange chromatography.

Other methods that are contemplated to be used for demonstrating or monitoring purity are, by way of example only, end group analysis, vapor pressure osmometry, cryoscopy/ebulliometry (freezing point depression/boiling point elevation), viscometry, small-angle X ray scattering, laser light scattering, optical absorption and scattering, ultracentrifugation, field flow fractionation, matrix-assisted laser desorption/ionization time-of-flight (MALDI-TOF) mass spectrometry, nuclear magnetic resonance spectrometry and crystallization.

### Method for Treating Cancer

Without limiting the scope of the compositions, methods and packaged product disclosed herein, the packaged product is used to treat several specific cancers or tumors. Cancer types include (some of which may overlap in scope), by way of example only, adrenal cortical cancer, anal cancer, aplastic anemia, bile duct cancer, bladder cancer, bone cancer, bone metastasis, adult CNS brain tumors, pediatric CNS brain metastases, brain metastases, breast cancer, Castleman Disease, cervical cancer, childhood Non-Hodgkin's lymphoma, colon and rectum cancer, endometrial cancer, esophagus cancer, Ewing's family of tumors, eye cancer, gallbladder cancer, gastrointestinal carcinoid tumors, gastrointestinal stromal tumors, gestational trophoblastic disease, hematological malignancies, Hodgkin's disease, Kaposi' sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, nonmelanoma skin cancer, stomach cancer, testicular cancer, thymus cancer, thyroid cancer, uterine sarcoma, vaginal cancer, vulvar cancer, and Waldenstrom's macroglobulinemia. In one embodiment, the cancers are selected from the group consisting of metastatic brain cancer, lung cancer, glioblastoma, lymphomas, leukemia, renal cell cancer (kidney cancer), head and neck cancer, breast cancer, prostrate cancer, and ovarian cancer. In one embodiment, the cancer has metastasized; in a further embodiment, the cancer has metastasized to the brain of the patient; in yet a further embodiment, the metastasized cancer is lung cancer.

Disclosed herein are methods, compositions, and packaged product useful for treating lung cancer comprising administration of the compositions of Formula (I). Treatment options for lung cancer include (which can be provided to a patient in conjunction with administration of the compositions of Formula (I)), by way of example only, surgery, immunotherapy, radiation therapy, chemotherapy, photodynamic therapy, or a combination thereof. Some possible surgical options for treatment of lung cancer are a segmental or wedge resection, a lobectomy, or a pneumonectomy. Radiation therapy may be external beam radiation therapy or brachytherapy. Also disclosed herein are methods, compositions, and packaged product useful for treating lung cancer that has metastasized to the brain of a patient, comprising administration of the compositions of Formula (I). In a further embodiment, the composition of Formula (I) is motexafin gadolinium. In a further embodiment, radiation therapy, including whole brain radiation therapy, is provided to the patient along with a therapeutically effective dose of motexafin gadolinium. In a further embodiment, administration of motexafin gadolinium with radiation therapy delays the progression of neurological deterioration and/or neurocognitive deterioration in the patient.

Disclosed herein are methods, compositions, and packaged product useful for treating CNS neoplasms comprising administration of the compositions of Formula (I). Treatment options for CNS neoplasms include (which can be provided to a patient in conjunction with administration of the compositions of Formula (I)), by way of example only, surgery, radiation therapy, immunotherapy, hyperthermia, gene therapy, chemotherapy, and combination of radiation and chemotherapy. Doctors also may prescribe steroids to reduce the swelling inside the CNS.

Disclosed herein are methods, compositions, and packaged product useful for treating kidney cancer comprising administration of the compositions of Formula (I). Kidney cancer (also called renal cell cancer or renal adenocarcinoma) is a disease in which malignant cells are found in the lining of tubules in the kidney. Treatment options for kidney cancer include (which can be provided to a patient in conjunction with administration of the compositions of Formula (I)), by way of example only, surgery, radiation therapy, chemotherapy and immunotherapy. Some possible surgical options to treat kidney cancer include, by way of example only, partial nephrectomy, simple nephrectomy and radical nephrectomy. Radiation therapy may be external beam radiation therapy or brachytherapy. Stem cell transplant may be used to treat kidney cancer.

Disclosed herein are methods, compositions, and packaged product useful for treating lymphoma comprising administration of the compositions of Formula (I). Treatment options for lymphoma include (which can be provided to a patient in conjunction with administration of the compositions of Formula (I)), by way of example only, chemotherapy, immunotherapy, radiation therapy and high-dose chemotherapy with stem cell transplant. Radiation therapy may be external beam radiation therapy or brachytherapy.

Disclosed herein are methods, compositions, and packaged product useful for treating breast cancer comprising administration of the compositions of Formula (I). Treatment options for breast cancer include (which can be provided to a patient in conjunction with administration of the compositions of Formula (I)), by way of example only, surgery, immunotherapy, radiation therapy, chemotherapy, endocrine therapy, or a combination thereof. A lumpectomy and a mastectomy are two possible surgical procedures available for breast cancer patients.

Disclosed herein are methods, compositions, and packaged product useful for treating ovarian cancer, comprising administration of the compositions of Formula (I). Treatment options for ovarian cancer include (which can be provided to a patient in conjunction with administration of the compositions of Formula (I)), by way of example only, surgery, immunotherapy, chemotherapy, hormone therapy, radiation therapy, or combinations thereof. Some possible surgical procedures include debulking, and a unilateral or bilateral oophorectomy and/or a unilateral or bilateral salpigectomy.

Disclosed herein are methods, compositions, and packaged product useful for treating cervical cancer, comprising administration of the compositions of Formula (I). Treatment options for cervical cancer include (which can be provided to a patient in conjunction with administration of the compositions of Formula (I)), by way of example only, surgery, immunotherapy, radiation therapy and chemotherapy. Some possible surgical options are cryosurgery, a hysterectomy, and a radical hysterectomy. Radiation therapy for cervical cancer patients includes external beam radiation therapy or brachytherapy.

Disclosed herein are methods, compositions, and packaged product useful for treating prostate cancer, comprising administration of the compositions of Formula (I). Treatment options for prostate cancer include (which can be provided to a patient in conjunction with administration of the compositions of Formula (I)), by way of example only, surgery, immunotherapy, radiation therapy, cryosurgery, hormone therapy, and chemotherapy. Possible surgical procedures to treat prostate cancer include, by way of example only, radical retropubic prostatectomy, a radical perineal prostatectomy, and a laparscopic radical prostatectomy. Some radiation therapy options are external beam radiation, including three dimensional conformal radiation therapy, intensity modulated radiation therapy, and conformal proton beam radiation therapy. Brachytherapy (seed implantation or interstitial radiation therapy) is also an available method of treatment for prostate cancer. Cryosurgery is another possible method used to treat localized prostate cancer cells. Hormone therapy, also called androgen deprivation therapy or androgen suppression therapy, may be used to treat prostate cancer. Several methods of this therapy are available including an orchiectomy in which the testicles, where 90% of androgens are produced, are removed. Another method is the administration of luteinizing hormone-releasing hormone (LHRH) analogs to lower androgen levels. The LHRH analogs available include leuprolide, goserelin, triptorelin, and histrelin. An LHRH antagonist may also be administered, such as abarelix. Treatment with an antiandrogen agent, which blocks androgen activity in the body, is another available therapy. Such agents include flutamide, bicalutamide, and nilutamide. This therapy is typically combined with LHRH analog administration or an orchiectomy, which is termed a combined androgen blockade (CAB). Chemotherapy may be appropriate where a prostate tumor has spread outside the prostate gland and hormone treatment is not effective. Anti-cancer drugs may be administered to slow the growth of prostate cancer, reduce symptoms and improve the quality of life.

Disclosed herein are methods, compositions, and packaged product useful for treating leukemia, comprising administration of the compositions of Formula (I). Treatment options for leukemia include (which can be provided to a patient in conjunction with administration of the compositions of Formula (I)), by way of example only, immunotherapy, radiation therapy, chemotherapy, bone marrow or peripheral blood stem cell transplantation, or a combination thereof. Radiation therapy includes external beam radiation and may have side effects. Anti-cancer drugs may be used in chemotherapy to treat leukemia. Monoclonal antibody therapy may be used to treat AML patients. Small molecules or radioactive chemicals may be attached to these antibodies before administration to a patient in order to provide a means of killing leukemia cells in the body. The monoclonal antibody, gemtuzumab ozogamicin, which binds CD33 on AML cells, may be used to treat AML patients unable to tolerate prior chemotherapy regimens. Bone marrow or peripheral blood stem cell transplantation may be used to treat AML patients. Some possible transplantation procedures are an allogenic or an autologous transplant.

Disclosed herein are methods, compositions, and packaged product useful for treating head and neck cancer, comprising administration of the compositions of Formula (I). Treatment options for head and neck cancer include (which can be provided to a patient in conjunction with administration of the compositions of Formula (I)), by way of example only, surgery, radiation, chemotherapy, combined modality therapy, gene therapy, either alone or in combination thereof.

### Method for Treating Cardiovascular Disease

Disclosed herein are methods, compositions, and packaged product useful for treating cardiovascular diseases. Cardiovascular disease has two main components: Diseases of the heart (cardio) and Diseases of the blood vessels (vascular).

### Coronary Artery Disease

These are diseases of the arteries that supply the heart muscle with blood. Sometimes known as CAD, coronary artery disease is the most common form of heart disease in industrialized nations and far and away the leading cause of heart attacks. Coronary artery disease generally means that blood flow through the arteries has become impaired. The most common way such obstructions develop is through a condition called atherosclerosis, a largely preventable type of vascular disease. These arteries, whose inner lining is normally smooth, can slowly become clogged with clumps of fats, cholesterol and other material, called atherosclerotic plaques. As a result, the supply of blood—with its oxygen and nutrients—going to the heart muscle is choked off (myocardial ischemia). Chest pain (angina pectoris) occurs, for instance, when the oxygen demand of the heart muscle exceeds the oxygen supply because of that narrowing in the coronary arteries. When the imbalance of oxygen supply lasts for more then a few minutes, heart muscle can begin to die, causing a heart attack (myocardial infarction). This may occur without symptoms (silent heart attack), especially in people with diabetes. In addition, the lack of blood, even briefly, can lead to serious disorders of the heart rhythm, known as arrhythmias or dysrhythmias. Coronary artery disease can even cause sudden death from an arrhythmia without any prior warning. A heart attack, for instance, can lead to congestive heart failure, and both of these conditions are types of cardiovascular disease. Other cardiovascular diseases are, by way of example only, cardiomyopathy, valvular heart disease, pericardial disease, congenital heart disease, and congestive heart failure.

Diseases of the blood vessels include, by way of example only, high blood pressure, aneurysms, occlusive artery disease, vasculitis, venus thrombosis.

### Peripheral Artery Disease (PAD)

In PAD, fatty deposits build up in the inner linings of the artery walls. These blockages restrict blood circulation, mainly in arteries leading to the kidneys, stomach, arms, legs and feet. In its early stages a common symptom is cramping or fatigue in the legs and buttocks during activity. Such cramping subsides when the person stands still. This is called “intermittent claudication.”People with PAD often have fatty buildup in the arteries of the heart and brain. Because of this association, people with PAD have a higher risk of death from heart attack and stroke. Treatments include, by way of example only, medicines to help improve walking distance, antiplatelet agents, and cholesterol-lowering agents (statins). Angioplasty or surgery may be necessary.

### Saphenous Vein Graft Disease

This term refers to the narrowing, either localized or diffuse, of the segment of the saphenous vein that has been used as a bypass graft. Serial follow-up studies of patients who have undergone bypass surgery have shown that up to 10 percent of vein grafts are occluded by the time of hospital discharge, which increases to 20 percent by the end of the first year after surgery. With time, there is continued development of disease and progression, so that by the end of 10 years only one-third of the vein grafts that were not occluded at one year are free of significant disease. A re-operation becomes necessary in about 20 percent of patients by 10 years. Treatments include, by way of example only, medical therapy, surgery, and angioplasty.

The packaged product disclosed herein can provide a beneficial effect for patients suffering from the aforementioned cardiovascular diseases, by administration of a compound of Formula (I) or a combination of administration of a compound of Formula (I) and at least one treatment identified in the foregoing paragraphs. In certain embodiments, the therapeutic agent is motexafin lutetium.

### Formulations, Routes of Administration, and Effective Doses

Another aspect disclosed herein relates to pharmaceutical packaged product compositions comprising a compound of Formula (I) and a pharmaceutically acceptable excipient. Such packaged pharmaceutical compositions can be used to treat cancer or cardiovascular disease in the methods as described herein.

The compounds of Formula (I) may be provided as a prodrug and/or may be allowed to interconvert to compound of Formula (I) in vivo after administration. The compounds of Formula (I) and/or its prodrug, or its pharmaceutically acceptable salts may be used in developing a formulation for use in the methods or packaged product disclosed herein. Further, in some embodiments, the compound may be used in combination with one or more other compounds or in one or more other forms.

The term “pharmaceutically acceptable salt” means those salts which retain the biological effectiveness and properties of the compounds disclosed herein, and which are not biologically or otherwise undesirable. For example, a pharmaceutically acceptable salt does not interfere with the beneficial effect of the compound disclosed herein in treating a cancer.

Typical salts include by way of example only, salts formed upon reaction of a compound of Formula (I) with hydrochloric acid, hydrobromic acid, phosphoric acid, nitric acid, sulfuric acid, methanesulfonic acid, p-toluenesulfonic acid, acetic acid, fumaric acid, succinic acid, lactic acid, mandelic acid, malic acid, citric acid, tartaric acid or maleic acid. In addition, if the packaged compounds disclosed herein contain a carboxy group or other acidic group, it may be converted into a pharmaceutically acceptable addition salt with inorganic or organic bases. Examples of suitable bases include by way of example only, sodium hydroxide, potassium hydroxide, ammonia, cyclohexylamine, dicyclohexyl-amine, ethanolamine, diethanolamine, tromethamine, meglumine and triethanolamine.

The compositions of Formula (I) may be administered in either single or multiple doses by any of the accepted modes of administration of agents having similar utilities, for example as described in those patents and patent applications incorporated by reference above, including intra-arterial injection, intravenously, intraperitoneally, parenterally, intramuscularly, or subcutaneously.

One mode for administration is parenteral, including, by way of example, by injection. The forms in which the compositions of Formula (I) may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parables, chlorobutanol, phenol, sorbic acid, thimerosal, and the like.

Sterile injectable solutions are prepared by incorporating the compositions of Formula (I) in the required amount in the appropriate solvent with various other ingredients as enumerated above, as required, followed by sterilization by filtration. Generally, dispersions are prepared by incorporating the various sterilized compositions of Formula (I) into a sterile vehicle which contains the basic dispersion medium and the required other ingredients from those enumerated above. In the case of sterile powders for the preparation of sterile injectable solutions, the methods of preparation are vacuum-drying and freeze-drying techniques, which yield a powder of the compositions of Formula (I) plus any additional desired ingredient from a previously sterile-filtered solution thereof.

The compositions are optionally formulated in a unit dosage form. The term “unit dosage form(s)” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampoule). The compositions of Formula (I) are effective over a wide dosage range and are generally administered in a pharmaceutically effective amount. For parenteral administration, from 10 to 700 mg of a composition of Formula (I), including about 350 mg. The amount of the compound actually administered will be determined by a physician, in the light of the relevant circumstances, including the condition to be treated, the chosen route of administration, the actual compound administered and its relative activity, the age, weight, and response of the individual patient, the severity of the patient's symptoms, and the like.

The specific dose will vary depending on the particular composition of Formula (I) chosen, the dosing regimen to be followed, and the particular therapeutic energy or agent with which it is administered, employing dosages within the range of about 0.01 mg/kg per treatment up to about 100 mg/kg per treatment, preferably about 0.1 mg/kg per treatment to about 50 mg/kg per treatment. Dosages of about 1.0 to 2.0 mg/kg to about 4.0 to 7.0 mg/kg, including 3.0 mg/kg, may be employed, although in some cases a maximum tolerated dose may be higher, for example about 10 mg/kg. There are specific differences in the most effective dosimetry depending on the apical ligands chosen, because of the wide range of properties available, such as solubilities, lipophilicity properties, lower toxicity, and improved stability.

### Administration for Photodynamic Therapy

By way of example, a composition of Formula (I), having lutetium as a metal in the texaphyrin, may be administered in solution, optionally in 5% mannitol USP. Dosages of about 1.0 to 2.0 mg/kg to about 4.0 to 7.0 mg/kg, including 3.0 mg/kg, are employed, although in some cases a maximum tolerated dose may be higher, for example about 5 mg/kg. The texaphyrin is administered by intravenous injection, followed by a waiting period of from as short a time as several minutes or about 3 hours to as long as about 72 or 96 hours (depending on the treatment being effected) to facilitate intracellular uptake and clearance from the plasma and extracellular matrix prior to the administration of photoirradiation.

Dose levels for certain uses may range from about 0.05 mg/kg to about 20 mg/kg administered in single or multiple doses (e.g., before each fraction of radiation). The lower dosage range would be applicable, for example, to intra-arterial injection or for impregnated stents.

The optimum length of time between administration of compositions of Formula (I) and light treatment can vary depending on the mode of administration, the form of administration, and the type of target tissue. Typically, the compositions of Formula (I) persists for a period of minutes to hours, depending on the composition of Formula (I), the formulation, the dose, the infusion rate, as well as the type of tissue and tissue size.

When employing photodynamic therapy, a target area is treated with light, for example at about 730±16.5 nm. After the photosensitizing composition of Formula (I) has been administered, the tissue being treated is photo irradiated at a wavelength similar to the absorbance of the composition of Formula (I), usually either about 440 to 540 nm or about 690 to 770 nm, or about 490 to 540 nm, or about 705 to 755 nm, or about 450 to 500 nm or about 725 to 735 nm. The light source may be a laser, a light-emitting diode, or filtered light from, for example, a xenon lamp; and the light may be administered topically, endoscopically, or interstitially (via, e.g., a fiber optic probe), or intra-arterially. In one embodiment, the light is administered using a slit-lamp delivery system. The fluence and irradiance during the photo irradiating treatment can vary depending on the type of tissue, depth of target tissue, and the amount of overlying fluid or blood. For example, a total light energy of about 100 J/cm2 can be delivered at a power of 200 mW to 250 mW, depending upon the target tissue.

Administration with Chemotherapeutic Drugs

Compositions of Formula (I) may be administered before, or after administration of one or more chemotherapeutic drugs. The composition of Formula (I) may be administered as a single dose, or it may be administered as two or more doses separated by an interval of time. The composition of Formula (I) may be administered concurrently with, or from about 1 minute to about 12 hours following administration of a chemotherapeutic drug, preferably from about 5 minutes to about 5 hours, more preferably about 4 to 5 hours. The dosing protocol may be repeated, from one to three times, for example. A time frame that has been successful in vivo is administration of a composition of Formula (I) about 5 minutes and about 5 hours after administration of a chemotherapeutic agent, with the protocol being performed once per week for three weeks. Administration may be intra-arterial injection, intravenous, intraperitoneal, intramuscular, subcutaneous, oral, topical, or via a device such as a stent.

Administering a composition of Formula (I) and a chemotherapeutic drug to the subject may be prior to, concurrent with, or following vascular intervention. The method may begin at a time roughly accompanying a vascular intervention, such as an angioplastic procedure, for example. Multiple or single treatments prior to, at the time of, or subsequent to the procedure may be used. “Roughly accompanying a vascular intervention” refers to a time period within the ambit of the effects of the vascular intervention. Typically, an initial dose of a composition of Formula (I) and chemotherapeutic drug will be within 6 to 12 hours of the vascular intervention, preferably within 6 hours thereafter. Follow-up dosages may be made at weekly, biweekly, or monthly intervals. Design of particular protocols depends on the individual subject, the condition of the subject, the design of dosage levels, and the judgment of the attending practitioner.

### Administration for Radiation Sensitization

Compositions of Formula (I), where the metal is gadolinium may be administered in a solution containing 2 to 3 mg/ml, optionally in 5% mannitol, USP/water (sterile and non-pyrogenic solution) and acetic acid. In further embodiments, the therapeutic agent is motexafin gadolinium; in further embodiments, the concentration of motexafin gadolinium is about 2.5 mg/mL. However, the actual doses given to the patient may vary from 0.1 mg of therapeutic agent per kg of patient, up to as high as about 29.0 mg/kg have been delivered, preferably about 3.0 to about 15.0 mg/kg (for volume of about 90 to 450 mL) may be employed, optionally with pre-medication using anti-emetics when dosing above about 6.0 mg/kg. The compound is administered via intravenous injection over about a 5 to 10 minute period, followed by a waiting period of about 2 to 5 hours to facilitate intracellular uptake and clearance from the plasma and extracellular matrix prior to the administration of radiation.

When employing whole brain radiation therapy, a course of 30 Gy in ten (10) fractions of radiation may be administered over consecutive days excluding weekends and holidays. In the treatment of brain metastases, whole brain megavolt radiation therapy is delivered with 60Co teletherapy or a 4 MV linear accelerator with isocenter distances of at least 80 cm, using isocentric techniques, opposed lateral fields and exclusion of the eyes. A minimum dose rate at the midplane in the brain on the central axis is about 0.5 Gy per minute.

Compositions of Formula (I) used as radiation sensitizers may be administered before, or at the same time as, or after administration of the ionizing radiation. The composition of Formula (I) may be administered as a single dose, as an infusion, or it may be administered as two or more doses separated by an interval of time. Where the composition of Formula (I) is administered as two or more doses, the time interval between the composition of Formula (I) administrations may be from about one minute to a number of days, from about 5 minutes to about 1 day, or about 4 to 5 hours. The dosing protocol may be repeated, from one to ten or more times, for example. Dose levels for radiation sensitization may range from about 0.05 mg/kg to about 20 mg/kg administered in single or multiple doses (e.g. before each fraction of radiation). The lower dosage range would be preferred for intra-arterial injection or for impregnated stents.

Administration may be intra-arterial injection, intravenous, intraperitoneal, intramuscular, or subcutaneous. In one aspect of the methods or packaged products for treating described herein, a patient having restenosis or at risk for restenosis is administered a dose of a composition of Formula (I) at intervals with each dose of radiation.

Administering a composition of Formula (I) to the subject may be prior to, concurrent with, or following vascular intervention, and the intervention is followed by radiation. The method may begin prior to, such as about 24 to 48 hours prior to, or at a time roughly accompanying vascular intervention, for example. Multiple or single treatments prior to, at the time of, or subsequent to the procedure may be used. “Roughly accompanying the vascular intervention” refers to a time period within the ambit of the effects of the vascular intervention. Typically, an initial dose of a composition of Formula (I) and radiation will be within 1 to 24 hours of the vascular intervention, preferably within about 5 to 24 hours thereafter. Follow-up dosages may be made at weekly, biweekly, or monthly intervals. Design of particular protocols depends on the individual subject, the condition of the subject, the design of dosage levels, and the judgment of the attending practitioner.

### Administration for Sonodynamic Therapy

The use of texaphyrins in sonodynamic therapy is described in U.S. patent application Ser. No. 09/111,148, which was converted to U.S. Provisional Application Ser. No. 60/155,256, from which a continuation was filed on Jan. 5, 2001, having U.S. patent application Ser. No. 09/755,824, now abandoned, which is incorporated herein by reference. Texaphyrin is administered before administration of the ultrasound. The texaphyrin may be administered as a single dose, or it may be administered as two or more doses separated by an interval of time. Parenteral administration is typical, including by intravenous and interarterial injection. Other common routes of administration can also be employed.

Ultrasound is generated by a focused array transducer driven by a power amplifier. The transducer can vary in diameter and spherical curvature to allow for variation of the focus of the ultrasonic output. Commercially available therapeutic ultrasound devices may be employed in the practice of such a method. The duration and wave frequency, including the type of wave employed may vary, and the preferred duration of treatment will vary from case to case within the judgment of the treating physician. Both progressive wave mode patterns and standing wave patterns have been successful in producing cavitation of diseased tissue. When using progressive waves, the second harmonic can advantageously be superimposed onto the fundamental wave. Types of ultrasound employed in such a method are ultrasound of low intensity, non-thermal ultrasound, i.e., ultrasound generated within the wavelengths of about 0.1 MHz and 5.0 MHz and at intensities between about 3.0 and 5.0 W/cm².

### EXAMPLES

### Example 1

### Container

In one embodiment, the container is a non-tinted borosilicate glass vial, USP Type I. The vial can hold a sufficient amount of a solution of Formula (I) to allow reliable administration of 50 mL of such a solution to a patient (which generally means the vial can hold 51-53 mL of solution). Further, such a vial has a suitable head space and an opening of 20 mm.

### Example 2

### Seal

In one embodiment, the seal is a one piece elastomeric bottle stopper composed of butyl rubber which forms a tight seal onto a glass bottle container housing Formula (I). In this embodiment, the stopper is a 20 mm flange type constructed from 4405/50 gray butyl rubber and laminated at the product contact area with a Teflon® film. Teflon® is fluorinated ethylene-propylene (FEP) applied as a film to the face of the stopper. The seal diameter is 20 mm and the seal is constructed of aluminum with a violet colored plastic Flip-Off® button.

### Example 3

### Outer Packaging

Each vial is packaged in an individual vial carton to afford protection from light. The cartons are made from 0.024 inch thick solid bleached sulfate paper and are coated on the outside. The base color of the carton exterior is bright white and cartons are imprinted with labeling text. The cartons are provided flat and are folded during packaging operations. One vial is placed per carton and the carton is folded or glued closed. The final dimensions of the folded and closed carton are 1¾ inches wide×1¾ inches deep×3¼ inches high.

### Example 4

### Container

A common clear borosilicate glass vial (USP Type I) with a nominal volume of 50 mL and a mouth opening of 20 mm was utilized. The use of amber glass was originally considered for light protection; however, it was shown that this type of glass did not provide adequate protection from wavelengths of light to which Formula (I) is sensitive. Vials of Formula (I) injection solution were protected from light by packaging them into cartons. The elastomeric closure, a 20 mm gray bromobutyl (4405/50) straight plug stopper, was selected based on low oxygen transmission values. In addition, a Teflon-faced stopper was selected to eliminate the direct contact of drug product with the elastomeric material and prevent staining of the closure by the intense green coloring of Formula (I). The stopper is held in place with a one-piece aluminum overseal.

Formula (I) injection solution is light sensitive. Protection from light is afforded by packaging each vial into an individual opaque vial carton. All of the vial labeling information is imprinted on the carton. A vial carton rather than an amber colored glass vial was chosen based on early formulation development showing that colored glass did not adequately protect the content of the vial from light. Preliminary photostability studies, after light exposure per ICH guidelines, showed that the chosen individual vial carton did afford light protection, see Table 4.1 below. In comparison, after light exposure, vials packaged in cartons showed virtually no change in free Gd content while unpackaged vials showed a marked increase in free gadolinium (Gd) content. An increase in free Gd is indicative of the degree of degradation in Formula (I) injection solution. For example, an increase of 10 μg/mL of free Gd is roughly equivalent to a 3% degradation of Formula (I) injection solution in a 2.5 mg/mL formulation.

TABLE 4.1
Results of Photostability samples, Batch A

| Sample | Gd content (μg/mL) | Degradation (estimated) |
|---|---|---|
| Boxed, control | 2.54 | — |
| Boxed, exposed | 3.75 | 0.4% |
| Unboxed, control | 2.56 | — |
| Unboxed, exposed | 16.99 | 4.3% |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purpose.

1-28. (canceled)

29. A solution comprising water, and a therapeutically-effective amount of a compound having the structure of Formula (I): 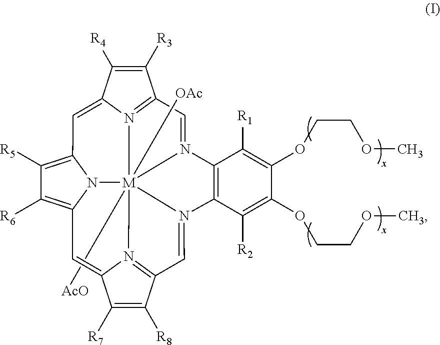
wherein:
M is a trivalent metal cation selected from the group consisting of Gd³⁺ and Lu³⁺;
R₃, R₄, R₅, R₆, R₇ and R₈ are independently H, OH, CₙH₍₂ₙ₊₁)O_{y} or OCₙH_{(2n+1)O}_{y};
wherein at least one of R₃, R₄, R₅, R₆, R₇, and R₈ is CₙH_{(2n+1)O}_{y}, having at least one hydroxy substituent;
R₁, R₂ are independently H or C₁-C₆ alkyl;
n is a positive integer from 1 to 11;
y is zero or a positive integer less than or equal to n;
wherein each x is identical and is selected from the group consisting of 2, 3, 4, 5, and 6; and a detectable level of free M in the solution less than 30 ppm.

30. The solution of claim 29, wherein the solution further comprises an acid.

31. The solution of claim 30, wherein the acid is acetic acid.

32. The solution of claim 30, wherein the solution has a pH between about 4.5 and about 5.5.

33. The solution of claim 29, wherein the solution further comprises an isotonic agent.

34. The solution of claim 33, wherein the isotonic agent is selected from the group consisting of saccharides, polyhydric alcohols, and dibasic sodium phosphate.

35. The solution of claim 34, wherein the isotonic agent is a polyhydric alcohol selected from the group consisting of mannitol, dextrose, sodium gluconate and sorbitol.

36. The solution claim 29, wherein the concentration of the compound of Formula (I) is between about 2.4 mg/mL and about 2.6 mg/mL.

37. The solution of claim 29, wherein M is Gd³⁺; R₄ and R₇ are C₃H₆OH; R₅ and R₆ are C₂H₅; R₃ and R₈ are CH₃; R₁ and R₂ are H; and x is 3.

38. The solution of claim 37, wherein the detectable level of free Gd³⁺ in the solution is less than 30 ppm for at least about 1 year.

39. The solution of claim 38, wherein the detectable level of free Gd³⁺ in the solution is less than 30 ppm for at least about 3 years.

40. The solution of claim 29, wherein the solution does not contain an oxidizing agent other than the compound of Formula (I) and oxygen.

41. The solution of claim 29, wherein the solution is suitable for administration to a human patient.

42. The solution of claim 29, wherein the solution is suitable for intravenous administration to a human patient.

43. The solution of claim 29, wherein the solution is suitable for intravenous administration to a human patient with cancer selected from the group consisting of renal cell cancer, lymphoma, leukemia, lung cancer and brain metastases arising from lung cancer.

44. The solution of claim 29, wherein the solution is sterile.

45. The solution of claim 29, wherein the solution is sterilized by filtration.
